(19) United States
(12) Patent Application Publication
(10) Pub. No.: US 2026/0310335 A1
(43) Pub. Date: Oct. 8, 2026

(54) USER INTERFACE FOR PARTICIPANT SELECTION DURING SURGICAL STREAMING

(71) Applicant: Digital Surgery Limited, London (GB)

(72) Inventors: Patrick deKlee, London (GB); Anthony J. Inwood, London (GB)

(21) Appl. No.: 19/478,920

(22) PCT Filed: Apr. 22, 2024
(86) PCT No.: PCT/EP2024/060891

§ 371 Date: Oct. 27, 2025

Related U.S. Application Data
(60) Provisional application No. 63/462,753, filed on Apr. 28, 2023.

(51) Int. Cl. G06F 3/0482; G06F 3/04845; G16H 40/67

(52) U.S. Cl. CPC G06F 3/0482; G06F 3/04845; G16H 40/67

(57) ABSTRACT

An aspect provides participant selection during surgical streaming of video and/or audio between an operating room and one or more external participants outside of the operating room. User interfaces and controls within the operating room can vary from external user interfaces and controls. A user within an operating room can invite participants and control whether an internal or external video stream is depicted in a main portion of a display. Each user can make individualized selections of content to be displayed on the main portion of each user display.

### BACKGROUND

The present disclosure relates in general to computing technology and relates more particularly to computing technology for multiple participant selection during surgical streaming.

Computer-assisted systems, particularly computer-assisted surgery (CAS) systems, can rely on video data digitally captured during a surgery in an operating room. Such video data can be stored and/or streamed. In some cases, the video data can be used within a system to augment a person's physical sensing, perception, and reaction capabilities. For example, such systems can effectively provide the information corresponding to an expanded field of vision, both temporal and spatial, that enables a person to adjust current and future actions based on the part of an environment not included in his or her physical field of view. Alternatively, or in addition, the video data, which can include or be accompanied by audio data captured by one or more microphones, can be stored and/or transmitted for several purposes such as archival, operational notes, training, post-surgery analysis, and/or patient consultation.

### SUMMARY

According to an aspect, a computer-implemented method includes displaying a first set of one or more video streams that are internal to an operating room on a first panel of an operating room display and displaying a second set of one or more video streams that are external to the operating room on a second panel of the operating room display. The method also includes detecting a selection of one of the one or more video streams of the second set as a selected video stream. The method further includes removing a current video stream displayed in a main portion of the operating room display as a removed video stream based on detecting the selection, and moving the selected video stream from the second panel to the main portion of the operating room display based on detecting the selection.

According to an aspect, a computer program product includes a memory device having computer executable instructions stored thereon, which when executed by one or more processors cause the one or more processors to perform operations including displaying two or more video streams that include at least one video stream internal to an operating room on a panel of a display and at least one video stream external to the operating room on the panel of the display. The operations also include detecting a selection of one of the two or more video streams as a selected video stream. The operations further include moving a current video stream displayed in a main portion of the display to the panel based on detecting the selection and retaining a surgeon video stream position pinned at a same location on the panel before and after moving the current video stream from the main portion of the display to the panel. The operations additionally include moving the selected video stream from the panel to the main portion of the display based on detecting the selection.

According to an aspect, a system includes an operating room display, a memory system, and a processing system coupled to the memory system and the operating room display. The processing system is configured to execute a plurality of instructions to display a first set of one or more video streams that are internal to an operating room on a first panel of the operating room display, display a second set of one or more video streams that are external to the operating room on a second panel of the operating room display, detect a selection of one of the one or more video streams of the first set or the second set as a selected video stream, and move the selected video stream to the main portion of the operating room display based on detecting the selection.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 depicts a computer-assisted surgery (CAS) system according to one or more aspects;

FIG. 2 depicts a surgical procedure system in accordance with one or more aspects;

FIG. 3 depicts a system for prediction generation that can be incorporated according to one or more aspects;

FIG. 4 depicts a user interface with multiple video streams in an operating room according to one or more aspects;

FIG. 5A depicts a user interface before participant stream selection internal to an operating room according to one or more aspects;

FIG. 5B depicts a user interface after participant stream selection internal to an operating room according to one or more aspects;

FIG. 6A depicts a user interface before participant stream selection external to an operating room according to one or more aspects;

FIG. 6B depicts a user interface after participant stream selection external to an operating room according to one or more aspects;

FIG. 7 depicts a flowchart of a method for video stream management of an operating room display internal to an operating room according to one or more aspects;

FIG. 8 depicts a flowchart of a method for video stream management of an operating room display external to an operating room according to one or more aspects; and

FIG. 9 depicts a computer system according to one or more aspects.

The diagrams depicted herein are illustrative. There can be many variations to the diagrams and/or the operations described herein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of the technical solutions described herein include systems and methods for video stream selection through one or more user interfaces during surgical procedures. As one example, a video streaming system can allow multiple participants external to an operating room to observe and interact with a surgeon or surgical team within the operating room. A user interface within the operating room can provide a surgeon or surgical team with the ability to invite one or more participants to observe the surgical procedure and interact through audio, video, and/or telestration. Participants outside of the operating room can use a different user interface that allows the participants to customize viewing preferences as well as interacting through audio, video, and/or telestration. Other interactions can occur through an interactive chat while streaming is active and comments which can be added during streaming or postoperatively to link with a recording of the surgical video. For example, surgical video can include an endoscopic view of a surgical procedure. Further, there can be multiple cameras or selectable points-of-view that capture the surgical procedure. In some aspects, the surgical video can be captured with overlaid content, such as structural identification using color and/or text overlays. The overlaid content can be merged with the surgical video or managed as another stream such that viewers may have an option of turning the overlaid content on or off. Further, the surgical team or other observers can generate a point-of-interest indicator while streaming is active to note occurrence of an event captured in at least one of the video streams. The point-of-interest indicator can be a timestamped marker or tag that aligns with a recording time of the surgical procedure. The point-of-interest indicator may be editable to add notes related to the event of interest during or after the surgical procedure is complete.

An operating room may contain a camera and microphone located on a central console and/or one or more cameras and microphones affixed (e.g., via a clip or other means) to medical personnel or objects in the operating room. In addition, or alternatively, one or more cameras and microphones can be attached or integrated into one or more devices in the operating room such as, but not limited to surgical tools, goggles, personal computers, smart watches, and/or smart phones. One or more cameras with microphones can be designated as providing a view of a surgeon or surgical team within the operating room. One or more surgical cameras can be incorporated with surgical tools, such as a laparoscopic or more generally, and endoscopic camera. Thus, while some resulting video feeds can include an audio portion, other video feeds may not include audio.

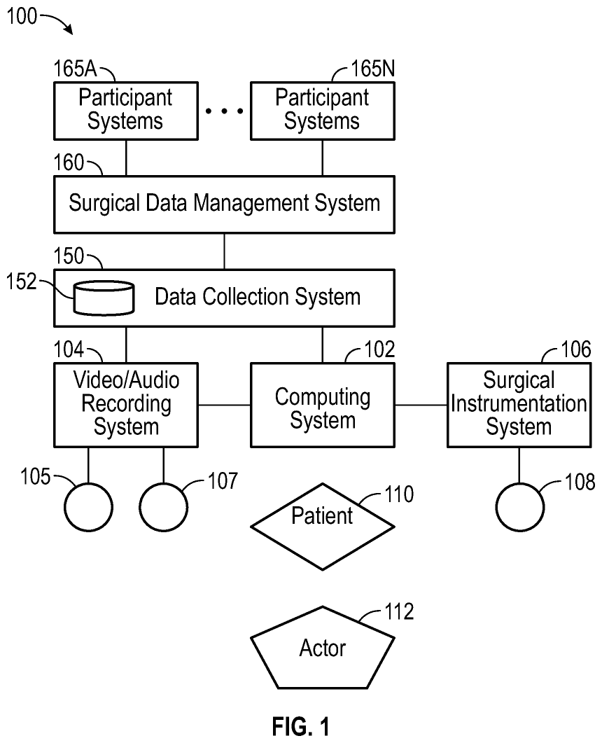

Turning now to FIG. 1, an example CAS system 100 is generally shown in accordance with one or more aspects. The CAS system 100 includes at least a computing system 102, a video/audio recording system 104, and a surgical instrumentation system 106. As illustrated in FIG. 1, an actor 112 can be medical personnel that uses the CAS system 100 to perform a surgical procedure on a patient 110. Medical personnel, or health care professionals, can be a surgeon, assistant, nurse, administrator, or any other actor that interacts with the CAS system 100 in a surgical environment. The surgical procedure can be any type of surgery, such as but not limited to open or laparoscopic hernia repair, laparoscopic cholecystectomy, robotic laparoscopic surgery, or any other surgical procedure with or without a robot. In other examples, actor 112 can be a surgeon, anesthesiologist, theatre nurse, technician, an administrator, an engineer, or any other such personnel that interacts with the CAS system 100. For example, actor 112 can record data from the CAS system 100, configure/update one or more attributes of the CAS system 100, review past performance of the CAS system 100, repair the CAS system 100, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g., closure). A “step” refers to the completion of a named surgical objective (e.g., hemostasis). During each step, certain surgical instruments 108 (e.g., forceps) are used to achieve a specific objective by performing one or more surgical actions.

The video/audio recording system 104 shown in FIG. 1 includes one or more cameras 105, such as operating room cameras, endoscopic cameras, etc. The cameras 105 capture video data of the surgical procedure being performed. The video/audio recording system 104 includes one or more video capture devices that can include cameras 105 placed in the surgical room to capture events surrounding (i.e., outside) the patient being operated upon. The video/audio recording system 104 further includes cameras 105 that are passed inside (e.g., endoscopic cameras) the patient 110 to capture endoscopic data. The endoscopic data provides video and images of the surgical procedure.

The video/audio recording system 104 also includes one or more microphones 107, which can be located on a central console, affixed (e.g., via a clip or other means) to medical personnel or objects in the operating room, and/or attached to or integrated into one or more devices in the operating room. Examples of devices in the operating room can include, but are not limited to surgical tools, video recorders, cameras, goggles, personal computers, smart watches, and/or smart phones. The microphones 107 capture audio data, and can be wired or wireless or a combination of both.

In exemplary aspects, the video data captured by the cameras 105 and the audio data captured by the microphones 107 can both include timestamps (or other indicia) that are used to correlate the video data and the audio data. The timestamps can be used to correlate, or synchronize, the sounds captured in the operating room with the images of the medical procedure performed in the operating room.

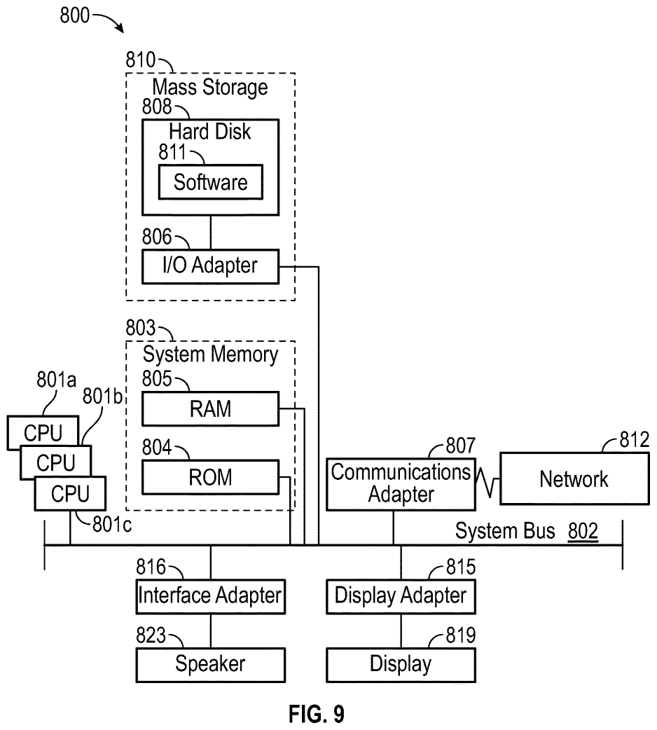

The computing system 102 includes one or more memory devices, one or more processors, and a user interface device, among other components. All or a portion of the computing system 102 shown in FIG. 1 can be implemented for example, by all or a portion of computer system 800 of FIG. 9. Computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed or has been performed earlier. Features can include structures such as anatomical structures, surgical instruments 108 in the captured video of the surgical procedure. Features can further include events such as phases, actions in the surgical procedure. Features that are detected can further include the actor 112 and/or patient 110. Based on the detection, the computing system 102, in one or more examples, can provide recommendations for subsequent actions to be taken by the actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, encoders, decoders, or any other type of machine learning model. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video/audio recording system 104. Alternatively, or in addition, the machine learning models use the surgical instrumentation data from the surgical instrumentation system 106. In yet other examples, the machine learning models use a combination of video data and surgical instrumentation data.

Additionally, in some examples, the machine learning models can also use audio data captured by the one or microphones 107 during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

After training, the one or more machine-learning models can then be used in real-time to process one or more data streams (e.g., video streams, audio streams, RFID data, etc.). The processing can include predicting and characterizing visualization modifications in images of a video of a surgical procedure based on one or more surgical phases, instruments, and/or other structures within various instantaneous or block time periods. The visualization can be modified to highlight the presence, position, and/or use of one or more structures. Alternatively, or in addition, the structures can be used to identify a stage within a workflow (e.g., as represented via a surgical data structure), predict a future stage within a workflow, etc.

In one or more examples, the machine learning models can detect surgical actions, surgical phases, anatomical structures, surgical instruments, activities, events, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery). In one or more examples, the machine learning models detect surgical phases based on detecting some of the features such as the anatomical structure, surgical instruments, etc.

A data collection system 150 can be employed to store the surgical data, including the video(s) captured during the surgical procedures and the audio data captured during the surgical procedure. The data collection system 150 includes one or more storage devices 152. The data collection system 150 can be a local storage system, a cloud-based storage system, or a combination thereof. Further, the data collection system 150 can use any type of cloud-based storage architecture, for example, public cloud, private cloud, hybrid cloud, etc. In some examples, the data collection system can use a distributed storage, i.e., the storage devices 152 are located at different geographic locations. The storage devices 152 can include any type of electronic data storage media used for recording machine-readable data, such as semiconductor-based, magnetic-based, optical-based storage media, or a combination thereof. For example, the data storage media can include flash-based solid-state drives (SSDs), magnetic-based hard disk drives, magnetic tape, optical discs, etc.

In one or more examples, the data collection system 150 can be part of the video/audio recording system 104, or vice-versa. In some examples, the data collection system 150, the video/audio recording system 104, and the computing system 102, can communicate with each other via a communication network, which can be wired, wireless, or a combination thereof. The communication between the systems can include the transfer of data (e.g., video data, audio data, instrumentation data, etc.), data manipulation commands (e.g., browse, copy, paste, move, delete, create, compress, etc.), data manipulation results, etc. In one or more examples, the computing system 102 can manipulate the data already stored/being stored in the data collection system 150 based on outputs from the one or more machine learning models, e.g., phase detection, structure detection, etc. Alternatively, or in addition, the computing system 102 can manipulate the data already stored/being stored in the data collection system 150 based on information from the surgical instrumentation system 106.

In one or more examples, the video captured by the video/audio recording system 104 is stored on the data collection system 150. In some examples, the computing system 102 curates parts of the video data being stored on the data collection system 150. In some examples, the computing system 102 filters the video captured by the video/audio recording system 104 before it is stored on the data collection system 150. Alternatively, or in addition, the computing system 102 filters the video captured by the video/audio recording system 104 after it is stored on the data collection system 150.

A surgical data management system 160 can provide access to portions of data captured in the data collection system 150, as well as data and records stored in other systems. Participant systems 165A-165N can access the surgical data management system 160 through one or more applications or secure web pages. Participant systems 165A-165N can include various types of computing devices, such as personal computers, laptop computers, tablet computers, mobile devices, smart appliances, and the like. The surgical data management system 160 can be a stand-alone application, module, and/or an extension of another system, including processing system and networking support hardware and software to support operation of the surgical data management system 160. Video, with or without audio, can pass through the data collection system 150 and surgical data management system 160 to support real-time streaming between users of the participant systems 165A-165N and one or more actors 112 through cameras 105 and/or microphones 107. Surgical instruments 108 can also be a source of streaming.

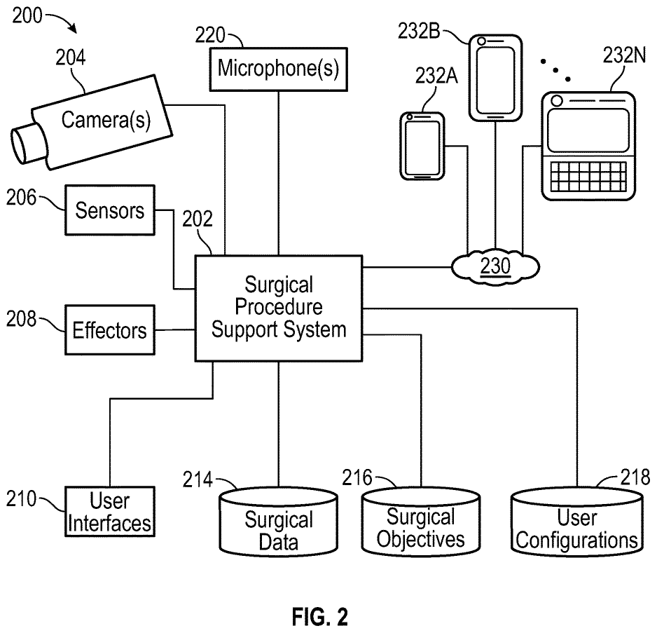

Turning now to FIG. 2, a surgical procedure system 200 is generally shown in accordance with one or more aspects. The example of FIG. 2 depicts a surgical procedure support system 202 that can include or may be coupled to the CAS system 100 of FIG. 1. The surgical procedure support system 202 can acquire image or video data using one or more cameras 204 (e.g., cameras 105 of FIG. 1). The surgical procedure support system 202 can also acquire audio data using one or more microphones 220 (e.g., microphones 107 of FIG. 1). The surgical procedure support system 202 can further interface with a plurality of sensors 206 and effectors 208. The sensors 206 may be associated with surgical support equipment and/or patient monitoring. The effectors 208 can be robotic components or other equipment (e.g., surgical instruments 108 of FIG. 1) controllable through the surgical procedure support system 202. The surgical procedure support system 202 can also interact with one or more user interfaces 210, such as various input and/or output devices. The surgical procedure support system 202 can store, access, and/or update surgical data 214 associated with a training dataset and/or live data as a surgical procedure is being performed on patient 110 of FIG. 1. The surgical procedure support system 202 can store, access, and/or update surgical objectives 216 to assist in training and guidance for one or more surgical procedures. User configurations 218 can track and store user preferences.

The surgical procedure support system 202 can also communicate with other systems through a network 230. For example, the surgical procedure support system 202 can communicate with other types of devices, such as a computing device 232A, 232B, . . . , 232N (e.g., a mobile phone, tablet computer, or laptop) through a network 230. As one example, user interfaces 210 may be connected to or integrated with the surgical procedure support system 202 by a local connection (e.g., within an operating room), while the mobile computing device 234 connects to the surgical procedure support system 202 via the network 230, which can extend over a substantial geographic area external to the operating room. The computing devices 232A-232N are examples of the participant systems 165A-165N of FIG. 1. Accordingly, the surgical data management system 160 of FIG. 1 can be interposed between the computing devices 232A-232N and the network 230 to manage data flow, streaming, and access constraints. Further, portions of the surgical data management system 160 can be executed locally by the computing devices 232A-232N and/or the surgical procedure support system 202.

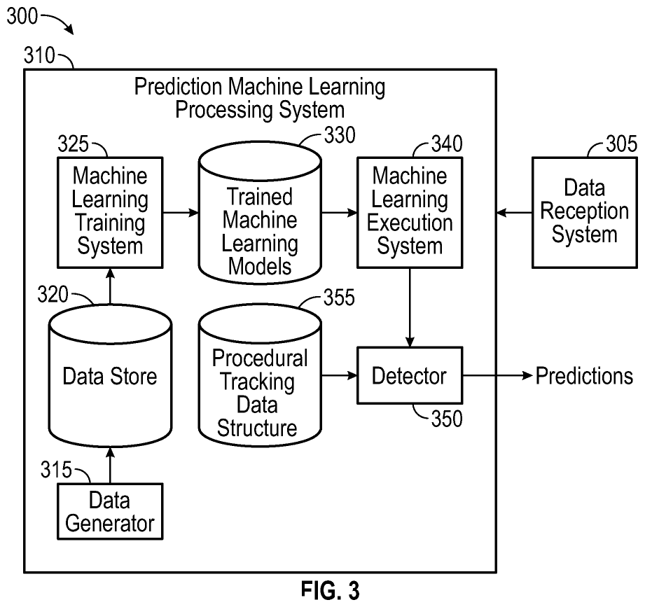

Turning now to FIG. 3, a system 300 for analyzing data that includes video data is generally shown according to one or more aspects. For example, the video data can be captured from video/audio recording system 104 of FIG. 1. The analysis can result in predicting surgical phases and structures (e.g., instruments, anatomical structures, etc.) in the video data using machine learning. System 300 can be the CAS system 100 of FIG. 1, or a part thereof in one or more examples. System 300 uses data streams in the surgical data to identify procedural states according to some aspects.

System 300 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed. Alternatively, or in addition, the data reception system 305 can receive or access surgical data in an offline manner, for example, by accessing data that is stored in the data collection system 150 of FIG. 1.

System 300 further includes a machine learning processing system 310 that processes the surgical data using one or more machine learning models to identify one or more features, such as surgical phase, instrument, anatomical structure, etc., in the surgical data. It will be appreciated that machine learning processing system 310 can include one or more devices (e.g., one or more servers), each of which can be configured to include part or all of one or more of the depicted components of the machine learning processing system 310. In some instances, a part or all of the machine learning processing system 310 is in the cloud and/or remote from an operating room and/or physical location corresponding to a part or all of data reception system 305. It will be appreciated that several components of the machine learning processing system 310 are depicted and described herein. However, the components are just one example structure of the machine learning processing system 310, and that in other examples, the machine learning processing system 310 can be structured using a different combination of the components. Such variations in the combination of the components are encompassed by the technical solutions described herein.

The machine learning processing system 310 includes a machine learning training system 325, which can be a separate device (e.g., server) that stores its output as one or more trained machine learning models 330. The machine learning models 330 are accessible by a machine learning execution system 340. The machine learning execution system 340 can be separate from the machine learning training system 325 in some examples. In other words, in some aspects, devices that “train” the models are separate from devices that “infer,” i.e., perform real-time processing of surgical data using the trained machine learning models 330.

Machine learning processing system 310, in some examples, further includes a data generator 315 to generate simulated surgical data, such as a set of virtual images, or record the video data from the video/audio recording system 104, to train the machine learning models 330. Data generator 315 can access (read/write) a data store 320 to record data, including multiple images and/or multiple videos. The images and/or videos can include images and/or videos collected during one or more procedures (e.g., one or more surgical procedures). For example, the images and/or video may have been collected by a user device worn by the actor 112 of FIG. 1 (e.g., surgeon, surgical nurse, anesthesiologist, etc.) during the surgery, a non-wearable imaging device located within an operating room, or an endoscopic camera inserted inside the patient 110 of FIG. 1. The data store 320 can be separate from the data collection system 150 of FIG. 1 in some examples. In other examples, the data store 320 can be part of the data collection system 150.

Each of the images and/or videos recorded in the data store 320 for training the machine learning models 330 can be defined as a base image and can be associated with other data that characterizes an associated procedure and/or rendering specifications. For example, the other data can identify a type of procedure, a location of a procedure, one or more people involved in performing the procedure, surgical objectives, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling. Localization can be performed using a variety of techniques for identifying objects in one or more coordinate systems.

The machine learning training system 325 uses the recorded data in the data store 320, which can include the simulated surgical data (e.g., set of virtual images) and actual surgical data to train the machine learning models 330. The machine learning model 330 can be defined based on a type of model and a set of hyperparameters (e.g., defined based on input from a client device). The machine learning models 330 can be configured based on a set of parameters that can be dynamically defined based on (e.g., continuous or repeated) training (i.e., learning, parameter tuning). Machine learning training system 325 can use one or more optimization algorithms to define the set of parameters to minimize or maximize one or more loss functions. The set of (learned) parameters can be stored as part of a trained machine learning model 330 using a specific data structure for that trained machine learning model 330. The data structure can also include one or more non-learnable variables (e.g., hyperparameters and/or model definitions).

Machine learning execution system 340 can access the data structure(s) of the machine learning models 330 and accordingly configure the machine learning models 330 for inference (i.e., prediction). The machine learning models 330 can include, for example, a fully convolutional network adaptation, an adversarial network model, an encoder, a decoder, or other types of machine learning models. The type of the machine learning models 330 can be indicated in the corresponding data structures. The machine learning model 330 can be configured in accordance with one or more hyperparameters and the set of learned parameters.

The one or more machine learning models 330, during execution, receive, as input, surgical data to be processed and subsequently generate one or more inferences according to the training. For example, the video data captured by the video/audio recording system 104 of FIG. 1 can include data streams (e.g., an array of intensity, depth, and/or RGB values) for a single image or for each of a set of frames (e.g., including multiple images or an image with sequencing data) representing a temporal window of fixed or variable length in a video. The video data that is captured by the video/audio recording system 104 can be received by the data reception system 305, which can include one or more devices located within an operating room where the surgical procedure is being performed. Alternatively, the data reception system 305 can include devices that are located remotely, to which the captured video data is streamed live during the performance of the surgical procedure. Alternatively, or in addition, the data reception system 305 accesses the data in an offline manner from the data collection system 150 or from any other data source (e.g., local or remote storage device).

The data reception system 305 can process the video and/or other data received. The processing can include decoding when a video stream is received in an encoded format such that data for a sequence of images can be extracted and processed. The data reception system 305 can also process other types of data included in the input surgical data. For example, the surgical data can include additional data streams, such as audio data, RFID data, textual data, measurements from one or more surgical instruments/sensors, etc., that can represent stimuli/procedural states from the operating room. The data reception system 305 synchronizes the different inputs from the different devices/sensors before inputting them in the machine learning processing system 310. In according to some aspects, audio data can also be used as a data source to generate predictions. Synchronization can be achieved by using a common reference clock to generate time stamps alongside each data stream. The clocks can be shared via network protocols or through hardware locking or through any other means. Such time stamps can be associated with any processed data format, such as, but not limited to text or other discrete data created from the audio signal. Additional synchronization can be performed by linking actions, events, or phase segmented that have been automatically processed from the raw signals using machine learning models. For example, text generated from an audio signal can be associated to specific phases of the procedure that are extracted from that audio or any other data stream signal. Text generated may be captured and/or displayed through a user interface.

The machine learning models 330, once trained, can analyze the input surgical data, and in one or more aspects, predict and/or characterize structures included in the video data included with the surgical data. The video data can include sequential images and/or encoded video data (e.g., using digital video file/stream formats and/or codecs, such as MP4, MOV, AVI, WEBM, AVCHD, OGG, etc.). The prediction and/or characterization of the structures can include segmenting the video data or predicting the localization of the structures with a probabilistic heatmap. In some instances, the one or more machine learning models include or are associated with a preprocessing or augmentation (e.g., intensity normalization, resizing, cropping, etc.) that is performed prior to segmenting the video data. An output of the one or more machine learning models can include image-segmentation or probabilistic heatmap data that indicates which (if any) of a defined set of structures are predicted within the video data, a location and/or position and/or pose of the structure(s) within the video data, and/or state of the structure(s). The location can be a set of coordinates in an image/frame in the video data. For example, the coordinates can provide a bounding box. The coordinates can provide boundaries that surround the structure(s) being predicted. The trained machine learning models 330, in one or more examples, are trained to perform higher-level predictions and tracking, such as predicting a phase of a surgical procedure and tracking one or more surgical instruments used in the surgical procedure.

While some techniques for predicting a surgical phase (“phase”) in the surgical procedure are described herein, it should be understood that any other technique for phase prediction can be used without affecting the aspects of the technical solutions described herein. In some examples, the machine learning processing system 310 includes a detector 350 that uses the machine learning models to identify a phase within the surgical procedure (“procedure”). Detector 350 uses a particular procedural tracking data structure 355 from a list of procedural tracking data structures. Detector 350 selects the procedural tracking data structure 355 based on the type of surgical procedure that is being performed. In one or more examples, the type of surgical procedure is predetermined or input by actor 112. The procedural tracking data structure 355 identifies a set of potential phases that can correspond to a part of the specific type of procedure.

In some examples, the procedural tracking data structure 355 can be a graph that includes a set of nodes and a set of edges, with each node corresponding to a potential phase. The edges can provide directional connections between nodes that indicate (via the direction) an expected order during which the phases will be encountered throughout an iteration of the procedure. The procedural tracking data structure 355 may include one or more branching nodes that feed to multiple next nodes and/or can include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state can indicate a complication (e.g., blood clots, clogged arteries/veins, etc.), pre-condition (e.g., lesions, polyps, etc.). In some examples, the machine learning models 330 are trained to detect an “abnormal condition,” such as hemorrhaging, arrhythmias, blood vessel abnormality, etc.

Each node within the procedural tracking data structure 355 can identify one or more characteristics of the phase corresponding to that node. The characteristics can include visual characteristics. In some instances, the node identifies one or more tools that are typically in use or availed for use (e.g., on a tool tray) during the phase. The node also identifies one or more roles of people who are typically performing a surgical task, a typical type of movement (e.g., of a hand or tool), etc. Thus, detector 350 can use the segmented data generated by machine learning execution system 340 that indicates the presence and/or characteristics of particular objects within a field of view to identify an estimated node to which the real image data corresponds. Identification of the node (i.e., phase) can further be based upon previously detected phases for a given procedural iteration and/or other detected input (e.g., verbal audio data that includes person-to-person requests or comments, explicit identifications of a current or past phase, information requests, etc.).

The detector 350 outputs the prediction associated with a portion of the video data that is analyzed by the machine learning processing system 310. The prediction is associated with the portion of the video data by identifying a start time and an end time of the portion of the video that is analyzed by the machine learning execution system 340. The prediction that is output can include an identity of a surgical phase, activity, or event as detected by the detector 350 based on the output of the machine learning execution system 340. Further, the prediction, in one or more examples, can include identities of the structures (e.g., instrument, anatomy, etc.) that are identified by the machine learning execution system 340 in the portion of the video that is analyzed. The prediction can also include a confidence score of the prediction. Various types of information in the prediction that can be output may include phases, actions, and/or events associated with a surgical procedure.

It should be noted that although some of the drawings depict endoscopic videos being analyzed, the technical solutions described herein can be applied to analyze video and image data captured by cameras that are not endoscopic (i.e., cameras external to the patient's body) when performing open surgeries (i.e., not laparoscopic surgeries). For example, the video and image data can be captured by cameras that are mounted on one or more personnel in the operating room, e.g., surgeon. Alternatively, or in addition, the cameras can be mounted on surgical instruments, walls, or other locations in the operating room.

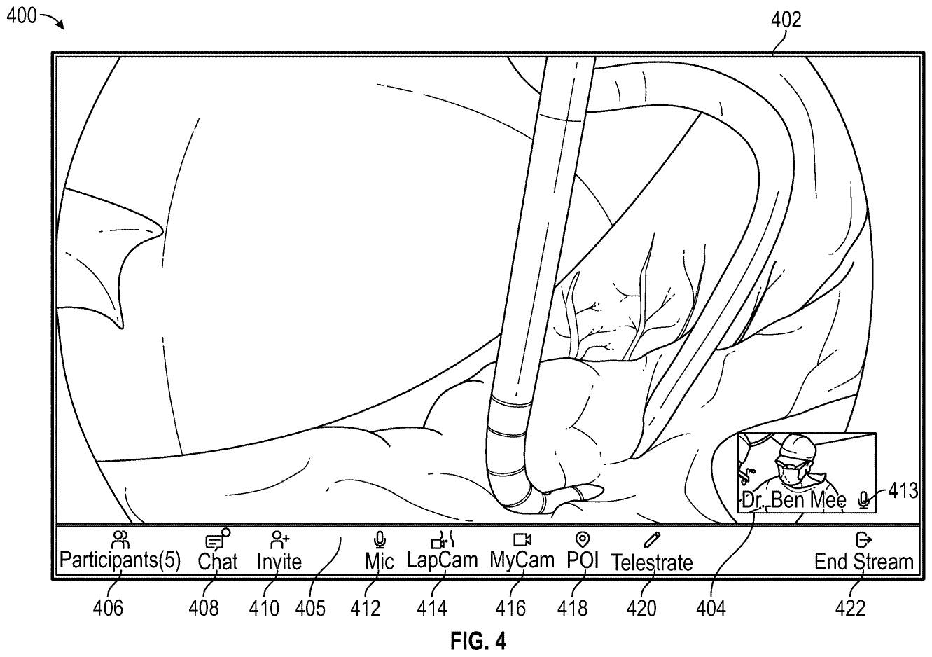

Turning now to FIG. 4, a user interface with multiple video streams in an operating room is depicting according to one or more aspects. The user interface of FIG. 4 is one example of a graphical user interface that can display content and receive input through the one or more user interfaces 210 of FIG. 2. In the example of FIG. 4, the user interface can be an operating room display 400 that displays a video stream in a main portion 402 and an inset portion 404. The video stream displayed in the main portion 402 can be selectable between in-operating room video streams and video streams that are external to the operating room. The inset portion 404 can include a surgeon video stream using a camera in the operating room that is oriented to view a surgeon or surgical team. In some aspects, the video source of content of the inset portion 404 is not selectable, such as where only a single camera input is available for the surgeon video stream. In other aspects, the video source of content of the inset portion 404 can be selectable between multiple sources. Further, display of the inset portion 404 can be toggled on or off. Additionally, the position of the inset portion 404 relative to the main portion 402 can be adjustable, for instance, to avoid obscuring content in the main portion 402.

User interface aspects of the operating room display 400 can include a control bar 405 with a plurality of selectable controls, such as a participants selector 406, a chat selector 408, an invite selector 410, a microphone control 412, a surgical camera control 414, a surgeon camera control 416, a point-of-interest (POI) selector 418, a telestration control 420, and an end stream control 422. The control bar 405 can be located below both the main portion 402 and the inset portion 404. Alternatively, the position of the control bar 405 can be located elsewhere on the operating room display 400, such as above the main portion 402 and the inset portion 404 or arranged as in a substantially vertical orientation to the left or right of the main portion 402. In some aspects, the position of the control bar 405 can be configurable depending on user preferences and/or other factors. Controls on the control bar 405 can be selected through a touch-sensitive screen, one or more buttons, or other input types, such as a mouse or trackball.

In aspects, selection of the participants selector 406 can result in transitioning the operating room display 400 to an adjusted state that opens one or more panels to view video streams of current participants in streaming and/or adjust the current content source for the main portion 402 of the operating room display 400. Selection of the chat selector 408 can open a chat interface to exchange messages and content between the participants and the surgeon or a sub-group thereof. The invite selector 410 can trigger sending of an electronic invitation for one or more parties to join as participants.

The microphone control 412 can operate as a mute button for one or more microphones in the operating room, where a mute or non-mute status indicator 413 can be overlaid on the inset portion 404 or elsewhere on the operating room display 400. In some aspects, the microphone control 412 can open a menu of options to adjust aspects of one or more of the microphones 107 of FIG. 1, such as mute/unmute one or more selected microphones, make gain adjustments, activate audio filtering, and/or other such actions.

The surgical camera control 414 can operate as a camera on/off control for a surgical camera, such as an endoscopic camera (e.g., a laparoscopic camera). In some aspects, surgical camera control 414 can open a menu of options to adjust aspects of one or more of the cameras 105 of FIG. 1, such as camera on/off for one or more selected cameras, make video quality adjustments, activate video filtering, activate video overlays, and/or other such actions. In the example of FIG. 4, the main portion 402 displays a surgical video stream from a laparoscopic camera. Thus, selecting the surgical camera control 414 can turn off or turn on the surgical video stream displayed in the main portion 402.

The surgeon camera control 416 can operate as a camera on/off control for a camera directed to capture a scene or point-of-view of one or more surgeons, such as camera(s) directed at one or more surgeons or attached to one or more surgeons. In some aspects, surgical camera control 414 can open a menu of options to adjust aspects of one or more of the cameras 105 of FIG. 1, such as camera on/off for one or more selected cameras, make video quality adjustments, activate video filtering, activate video overlays, and/or other such actions. In the example of FIG. 4, the main portion 402 displays a surgeon video stream in the inset portion 404. Accordingly, selecting the surgeon camera control 416 can turn off or turn on the surgeon video stream displayed in the inset portion 404.

The POI selector 418 can generate an indication of a POI associated with at least one video stream to note occurrence of an event captured in at least one of the video streams. The POI can be a time stamp that aligns with a clock or time since recording was initiated. Further, the selection of a POI through the POI selector 418 can result in a pop-up message appearing that is visible to the participants. The POI may also include an option to select a POI note from a prepopulated list of options or manually enter text.

The telestration control 420 can allow a user to electronically draw on a video stream as an overlay. For example, a surgeon may draw freehand using a stylus, a finger, a mouse, or other such input to highlight a feature depicted in the main portion 402. Further, where remote telestration is enabled, a remote participant can also have the ability to make telestrations that are visible to the surgeon and other participants. In some aspects, the telestrations can remain active for a predetermined period of time (e.g., 2 to 15 seconds) before automatically erasing. When a series of telestrations is performed, the time for auto-erase can be set and reset based on the most recent telestration. Further, the auto-erase count down can be based on when a first telestration begins through user input or a most recent telestration ends through user input. For instance, as one example, if the predetermined period of time is set to 5 seconds, the countdown of 5 seconds to zero can begin upon a first drawing action in a sequence. As another example, the countdown of 5 seconds to zero can be reset upon each subsequent telestration action. That is, if a user draws a first shape through telestration at time T and then draws a second shape 3 seconds later (e.g., T+3 seconds), the countdown timer can be based off the more recently drawn second shape, resulting in the first and second shape being automatically erased at time T+8 second in this example. Further, selection of the telestration control 420 can open a menu of options, such as various colors, line thickness, and behavior differences (e.g., auto-erase or manual erase).

The end stream control 422 can terminate the streaming session for all participants and the surgeon. In some aspects, selection of the end stream control 422 can close the operating room display 400 and prevent audio and video from the operating room being streamed to the participants. In some aspects, the participants may still be able to participate in a shared streaming session with each other, for instance to review the surgical procedure, share notes, and the like, even after the streaming from the operating room has ended.

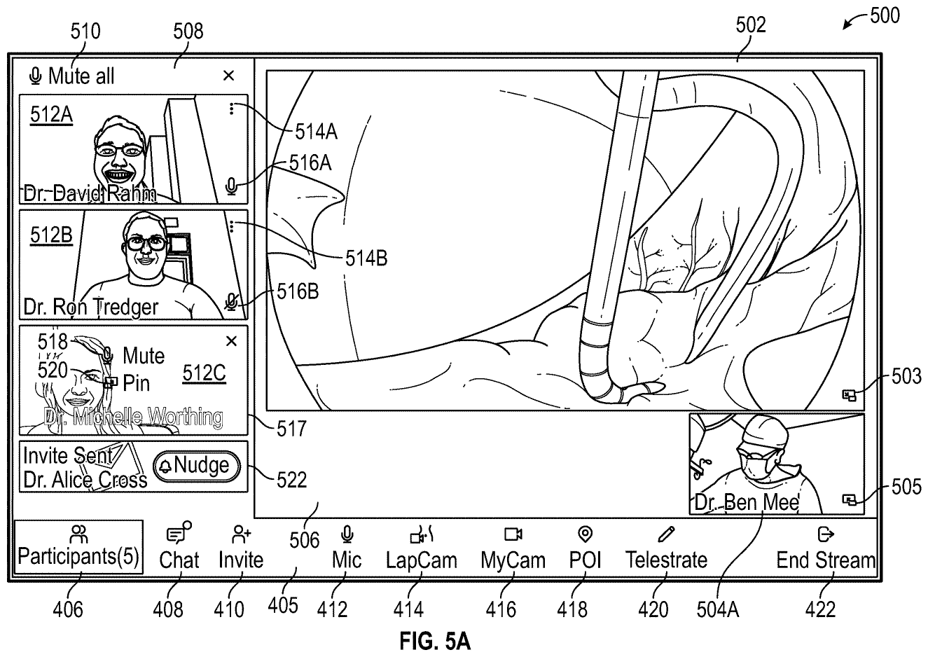

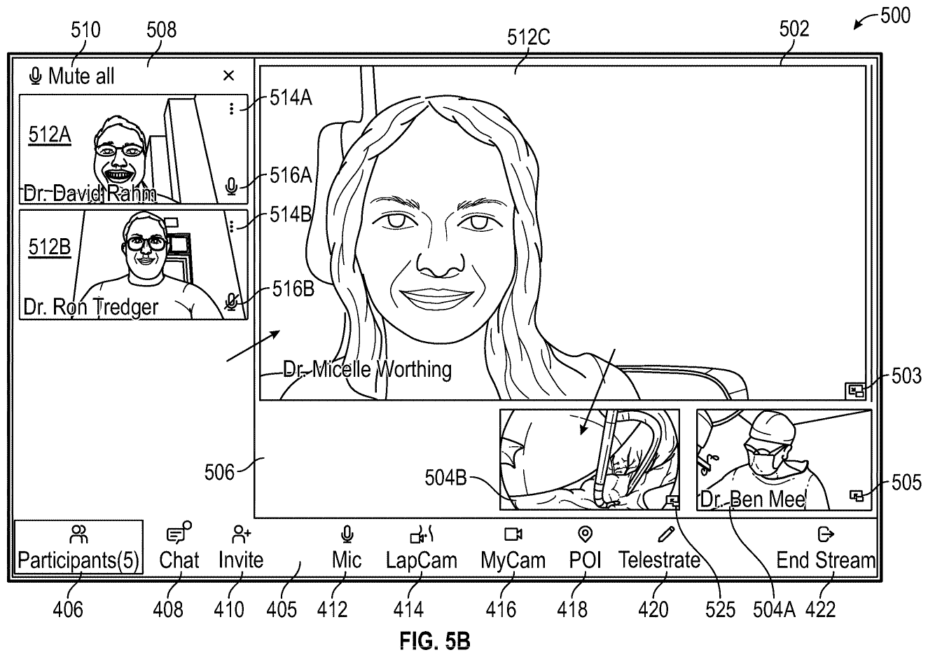

FIG. 5A depicts a user interface before participant stream selection internal to an operating room according to one or more aspects. In the example of FIG. 5A, upon an in-operating room user selecting the participants selector 406, a first set of one or more video streams 504A (e.g., up to 504N (not depicted)) that are internal to the operating room can be displayed on a first panel 506 of an operating room display 500, and a second set of one or more video streams 512A, 512B, 512C (e.g., up to 512N (not depicted)) that are external to the operating room can be displayed on a second panel 508 of the operating room display 500. Opening the first panel 506 and the second panel 508 can result in a size reduction of main portion 502 as compared to main portion 402 of FIG. 4. The video stream in the main portion 502 can include a pinning control 503 that returns the displayed video stream to the appropriate panel. In the example of FIG. 5A, the current video stream in the main portion 502 is a surgical video stream and thus would be returned to the first panel 506 upon selecting the pinning control 503. Similarly, the video stream 504A is a surgeon video stream with a pinning control 505 that upon selection while of the first panel 506 would result in moving the video stream 504A to the main portion 502 and removing the video stream 504A from the first panel 506. If a current video stream occupies the main portion 502, selection of pinning control 505 can result in both moving the current video stream to the appropriate panel (e.g., returning a surgical video stream to the first panel or returning a participant video stream 512 to the second panel 508), while also moving the video stream 504A to the main portion 502.

Selection of one of the video streams 512A-512C for pinning to the main portion 502 can be performed through a multiple step process is some aspects. For example, an expansion command 514A, 514B can be selected to open an expanded command menu 517 for a selected video stream, such as video stream 512C. The expanded command menu 517 can appear as a translucent overlay of the video stream 512C. Example of commands on the expanded command menu 517 can include a mute command 518 to turn an audio stream of the associated user on or off, a pinning control 520 to expand the video stream 512C into the main portion 502, and other commands.

In some aspects, the second panel 508 can include other controls, such as a mute all control 510 that inhibits an audio feed of the one or more participants (e.g., all participants) of the second panel 508 through a user interface. The mute status of each participant can be seen visually through a mute status indicator 516A, 516B associated with video streams 512A, 512B respectively.

FIG. 5A also illustrates an example of an invitation status 522 that can be depicted when the invite selector 410 is used to invite a new participant. The invitation status 522 may also include a nudge control which can trigger sending one or more reminders to the party invited to join the current streaming session.

FIG. 5B depicts a user interface after participant stream selection internal to an operating room according to one or more aspects. In the example of FIG. 5B, after a user in the operating room selects video stream 512C to be pinned to the main portion 502 (e.g., by selecting pinning control 520 of FIG. 5A), the current video stream displayed in the main portion 502 of the operating room display 500 in FIG. 5A is removed as a removed video stream based on detecting the selection, and the selected video stream (e.g., video stream 512C) is moved from the second panel 508 to the main portion 502 of the operating room display 500 based on detecting the selection. Selecting the pinning control 503 in the main portion 502 in the example of FIG. 5B would result in moving the video stream 512C back to the second panel 508.

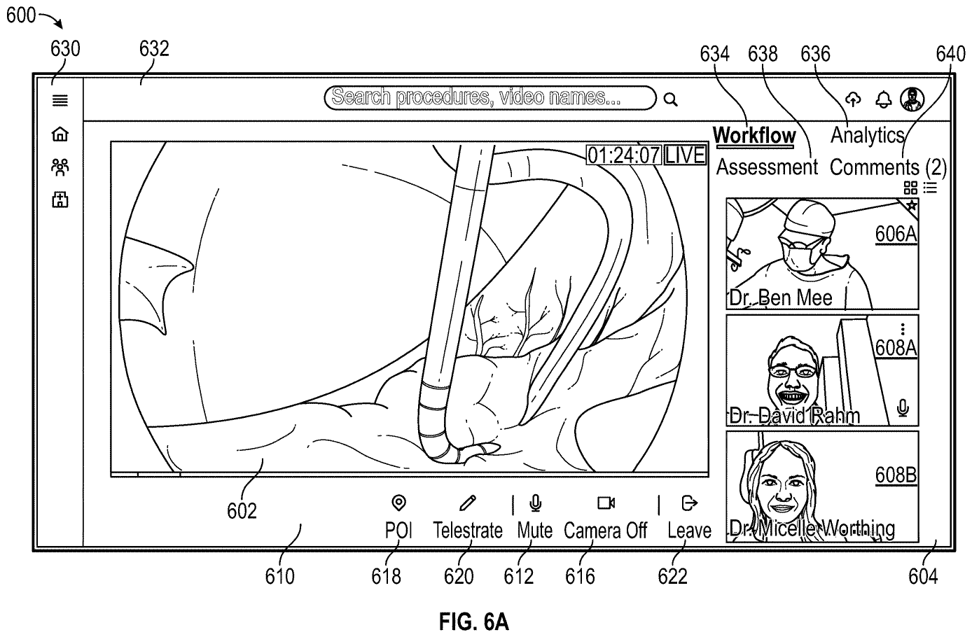

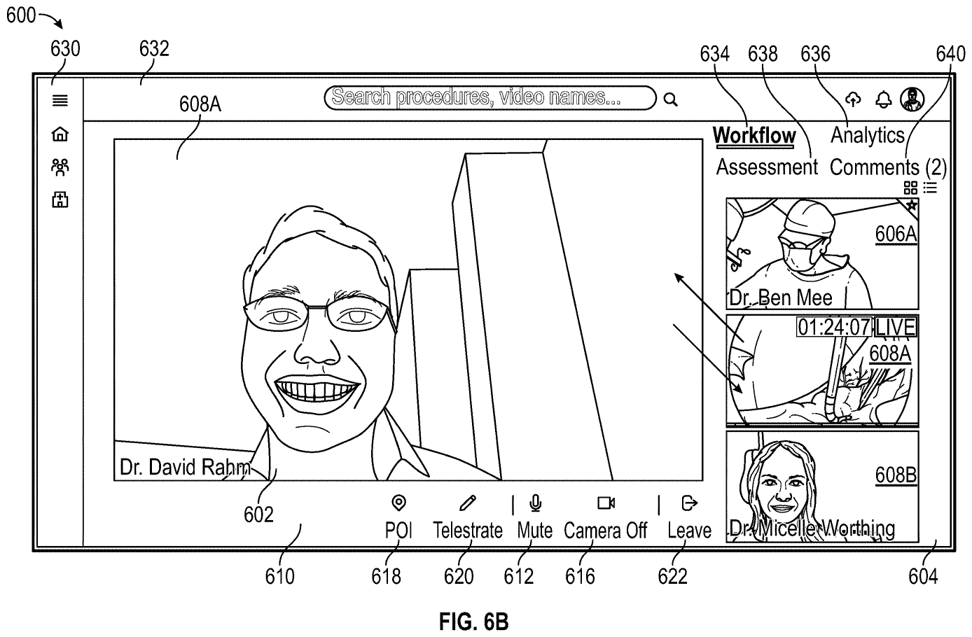

FIG. 6A depicts a user interface before participant stream selection external to an operating room according to one or more aspects. In the example of FIG. 6A, two or more video streams can be displayed including at least one video stream internal (e.g., video stream 606) to an operating room on a panel 604 of a display 600 and at least one video stream external (e.g., 608A, 608B) to the operating room on the panel 604 of the display 600. Any of the video streams can be selected for display in a main portion 602 of the display 600. For example, one of the two or more video streams (e.g., video streams 606, 608A, 608B) can be selected as a selected video stream. Selection can be based on a dragging action, a clicking action (e.g., double clicking), or a target location selection (e.g., an icon), for instance. A current video stream displayed in the main portion 602 of the display 600 can be moved to the panel 604 based on detecting the selection. Where the selected video stream is not a surgeon video stream (e.g., video stream 606A), the surgeon video stream position can be pinned and retrained at a same location on the panel 604 before and after moving the current video stream from the main portion 602 of the display 600 to the panel 604. For example, upon selecting video stream 608A, the selected video stream 608A can be moved from the panel 604 to the main portion 602 and the current video stream (e.g., video stream 606B) can be moved back to the panel 604 as depicted in the example of FIG. 6B.

Relative positioning of video streams (e.g., video streams 606A, 606B, 608A, 608B) on the panel 604 can be modified based on one or more placement rules. Relative placement of each stream on the panel 604 can vary for each user. For example, video streams internal to an operating room may be given higher priority for display than video streams external to the operating room. As such, a surgeon video stream (e.g., video stream 606A) may remain at the uppermost portion of the panel 604 as new video streams are added. New video streams or video streams returning from the main portion 602 can be place below or in close proximity to the surgeon video stream. Participant video streams for users external to the operating room can be placed below the in-operating room video streams. Further, user of the display 600 may remain in the panel 604 from the user perspective, while other users can be added or shifted in position according to placement rules. For instance, a currently or most recently speaking user may be given a higher priority placement (e.g., closer to the top of panel 604) than users who have not recently spoken. As another example, a user performing a screen share or telestration may be given a higher priority placement. As a further example, video streams more recently appearing in the main portion 602 may be given a higher priority placement. Where participants turn off their respective cameras, audio-only streams may be given lower placement priority. Other placement rule variations are contemplated.

In aspects, the user interface of display 600 can include a number of other controls. For example, the display 600 can include a control bar 610 with a plurality of selectable controls, such as a mute control 612, a camera control 616, a POI selector 618, a telestration control 620, and an end control 622. The mute control 612 can turn a user microphone on or off. The camera control 616 can turn a user camera on or off. The POI selector 618 can function similar to the POI selector 418 of FIGS. 5A, 5B. The telestration control 620 can function similar to the telestration control 420 of FIGS. 5A, 5B. The end control 622 can end a current session of a user. Further examples of controls on the user interface of display 600 can include an selection bar 630 that can trigger various actions, such as displaying a menu, navigating to a home interface, contacting other users, viewing facility information, and other such actions. As another example, a tool bar 632 can support searches, content upload, notification management, user account management (e.g., logon/logoff/profile editing), and other such actions. Additionally, other user interfaces can be selectable, such as a workflow interface 634, an analysis interface 636, an assessment interface 638, a comments interface 640, and other such interfaces.

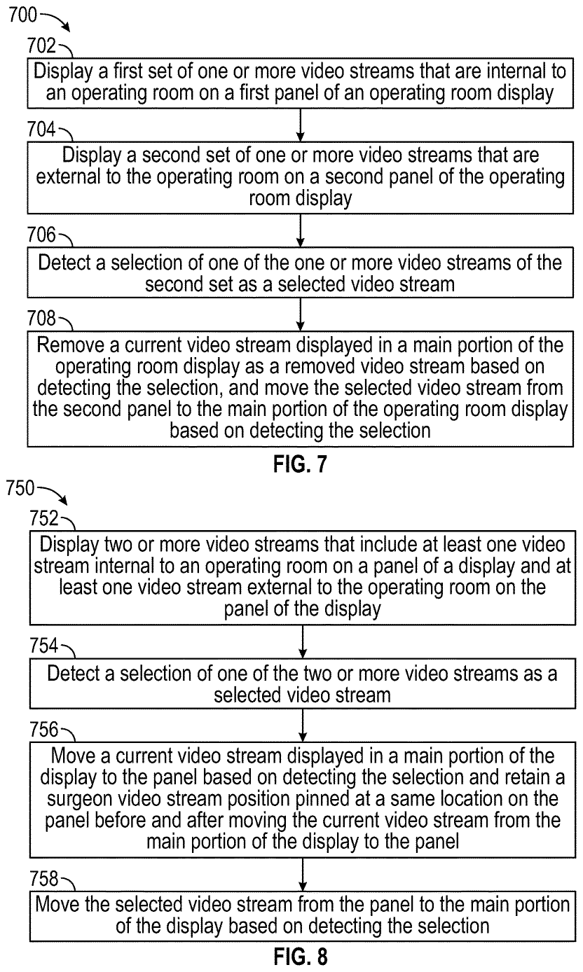

Turning now to FIG. 7, a flowchart of a method 700 for video stream management of an operating room display internal to an operating room is generally shown in accordance with one or more aspects. All or a portion of method 700 can be implemented, for example, by all or a portion of CAS system 100 of FIG. 1, surgical procedure system 200, and/or computer system 800 of FIG. 9.

At block 702, a first set of one or more video streams 504 that are internal to an operating room can be displayed on a first panel 506 of an operating room display 500. At block 704, a second set of one or more video streams 512 that are external to the operating room on a second panel 508 of the operating room display 500. At block 706, a selection of one of the one or more video streams 512 of the second set can be detected as a selected video stream. At block 708, a current video stream displayed in the main portion 502 of the operating room display 500 can be removed as a removed video stream based on detecting the selection, and the selected video stream can be moved from the second panel 508 to the main portion 502 of the operating room display 500 based on detecting the selection. Other types of selections and movements can be performed with respect to the panels 506, 508. For example, a surgeon video stream can be moved to the main portion 502 or other in-operating room video streams. Further, one of the participants may switch from a camera-based video stream to a shared-screen based video feed, and a user in the operating room can select the shared-screen based video feed to be displayed in the main portion 502. Other variations are contemplated.

The processing shown in FIG. 7 is not intended to indicate that the operations are to be executed in any particular order or that all of the operations shown in FIG. 7 are to be included in every case. Additionally, the processing shown in FIG. 7 can include any suitable number of additional operations.

FIG. 8 depicts a flowchart of a method 750 for video stream management of an operating room display external to an operating room according to one or more aspects. All or a portion of method 750 can be implemented, for example, by all or a portion of CAS system 100 of FIG. 1, surgical procedure system 200, and/or computer system 800 of FIG. 9.

At block 752, two or more video streams that include at least one video stream (e.g., video streams 606A, 606B) internal to an operating room can be displayed on a panel 604 of a display 600, and at least one video stream (e.g., 608A, 608B) external to the operating room can be displayed on the panel 604 of the display 600. At block 754, a selection of one of the two or more video streams can be detected as a selected video stream. At block 756, a current video stream displayed in a main portion 602 of the display 600 can be moved to the panel 604 based on detecting the selection, and a surgeon video stream position (e.g., an uppermost/upper-left position) can be pinned and retained at a same location on the panel 604 before and after moving the current video stream from the main portion 602 of the display 600 to the panel 604. At block 758, the selected video stream can be moved from the panel 604 to the main portion 602 of the display 600 based on detecting the selection. Other variations are contemplated.

The processing shown in FIG. 8 is not intended to indicate that the operations are to be executed in any particular order or that all of the operations shown in FIG. 8 are to be included in every case. Additionally, the processing shown in FIG. 8 can include any suitable number of additional operations.

Turning now to FIG. 9, a computer system 800 is generally shown in accordance with an aspect. The computer system 800 can be an electronic computer framework comprising and/or employing any number and combination of computing devices and networks utilizing various communication technologies, as described herein. The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer-executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 9, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801 can be any type of circuitry capable of executing instructions. The processors 801, also referred to as processing circuits, are coupled via a system bus 802 to a system memory 803 and various other components. The system memory 803 can include one or more memory devices, such as read-only memory (ROM) 804 and a random-access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 9.

Additional input/output devices are shown as connected to the system bus 802 via a display adapter 815 and an interface adapter 816. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 9, the computer system 800 includes processing capability in the form of the processors 801, and storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 9 is not intended to indicate that the computer system 800 is to include all of the components shown in FIG. 9. Rather, the computer system 800 can include any appropriate fewer or additional components not illustrated in FIG. 9 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects. Various aspects can be combined to include two or more of the aspects described herein.

According to an aspect, a computer-implemented method includes displaying a first set of one or more video streams that are internal to an operating room on a first panel of an operating room display, displaying a second set of one or more video streams that are external to the operating room on a second panel of the operating room display, detecting a selection of one of the one or more video streams of the second set as a selected video stream, and removing a current video stream displayed in a main portion of the operating room display as a removed video stream based on detecting the selection, and moving the selected video stream from the second panel to the main portion of the operating room display based on detecting the selection.

According to an aspect, the first set of one or more video streams can include at least one surgeon video stream and at least one endoscopic video stream.

According to an aspect, the method can include moving the removed video stream to the first panel based on determining that the removed video stream is associated with the first set of one or more video streams that are internal to the operating room.

According to an aspect, the method can include moving the removed video stream to the second panel based on determining that the removed video stream is associated with the second set of one or more video streams that are internal to the operating room.

According to an aspect, the method can include detecting a next selection of one of the one or more video streams that are internal to the operating room as the selected video stream and moving the selected video stream from the first panel to the main portion of the operating room display based on detecting the next selection.

According to an aspect, the method can include opening the second panel based on detecting a first user selection to view one or more participants through a user interface.

According to an aspect, the method can include closing the second panel based on detecting a second user selection to stop viewing one or more participants through the user interface.

According to an aspect, the method can include opening the first panel based on detecting the first user selection to view one or more participants through the user interface.

According to an aspect, the method can include where the second panel includes a mute all control that inhibits an audio feed of the one or more participants of the second panel through the user interface.

According to an aspect, the method can include where the selection of one of the one or more video streams of the second set comprises detecting a first selection of an expanded command menu and a second selection of a pin control on the expanded command menu.

According to an aspect, a computer program product can include a memory device having computer executable instructions stored thereon, which when executed by one or more processors cause the one or more processors to perform operations including displaying two or more video streams that include at least one video stream internal to an operating room on a panel of a display and at least one video stream external to the operating room on the panel of the display. The operations can also include detecting a selection of one of the two or more video streams as a selected video stream. The operations can further include moving a current video stream displayed in a main portion of the display to the panel based on detecting the selection and retaining a surgeon video stream position pinned at a same location on the panel before and after moving the current video stream from the main portion of the display to the panel. The operations can additionally include moving the selected video stream from the panel to the main portion of the display based on detecting the selection.

According to an aspect, the at least one video stream internal to the operating room can include at least one endoscopic video stream accessible by the one or more processors.

According to an aspect, the operations performed by the one or more processors can include maintaining the at least one endoscopic video stream at a position on the panel that has a higher viewing priority than the at least video stream external to the operating room on the panel.

According to an aspect, the operations performed by the one or more processors can include adjusting a relative position of one or more of the at least one video stream external to the operating room on the panel based on one or more placement rules.

According to an aspect, the selection can be separately controlled by each user interacting with the display through execution of the operations by the one or more processors.

According to an aspect, the operations performed by the one or more processors can include detecting a point-of-interest selection through at least one user interface of one of a plurality of users, and generating an indication of the point-of-interest associated with at least one video stream of the display to note occurrence of an event captured in at least one of the video streams.

According to an aspect, a system includes an operating room display, a memory system, and a processing system coupled to the memory system and the operating room display. The processing system is configured to execute a plurality of instructions to display a first set of one or more video streams that are internal to an operating room on a first panel of the operating room display, display a second set of one or more video streams that are external to the operating room on a second panel of the operating room display, detect a selection of one of the one or more video streams of the first set or the second set as a selected video stream, and move the selected video stream to the main portion of the operating room display based on detecting the selection.

According to an aspect, the first panel can be arranged horizontally on the operating room display and the second panel is arranged vertically on the operating room display.

According to an aspect, the first panel and the second panel can be opened based on detecting a user selection to view one or more participants through a user interface.

According to an aspect, the selection of one of the one or more video streams of the first set can include a single user action, and the selection of one of the one or more video streams of the second set comprises at least two user actions.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. The wireless network(s) can include, but is not limited to fifth generation (5G) and sixth generation (6G) protocols and connections. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, high-level languages such as Python, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), graphics processing units (GPUs), general-purpose microprocessors, application-specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
displaying a first set of one or more video streams that are internal to an operating room on a first panel of an operating room display;
displaying a second set of one or more video streams that are external to the operating room on a second panel of the operating room display;
detecting a selection of one of the one or more video streams of the second set as a selected video stream; and
removing a current video stream displayed in a main portion of the operating room display as a removed video stream based on detecting the selection, and moving the selected video stream from the second panel to the main portion of the operating room display based on detecting the selection.

2. The method of claim 1, wherein the first set of one or more video streams comprises at least one surgeon video stream and at least one endoscopic video stream.

3. The method of claim 1 or claim, further comprising:
moving the removed video stream to the first panel based on determining that the removed video stream is associated with the first set of one or more video streams that are internal to the operating room.

4. The method of any preceding claim, further comprising:
moving the removed video stream to the second panel based on determining that the removed video stream is associated with the second set of one or more video streams that are internal to the operating room.

5. The method of any preceding claim, further comprising.
detecting a next selection of one of the one or more video streams that are internal to the operating room as the selected video stream; and
moving the selected video stream from the first panel to the main portion of the operating room display based on detecting the next selection.

6. The method of any preceding claim, further comprising:
opening the second panel based on detecting a first user selection to view one or more participants through a user interface.

7. The method of claim 6, further comprising:
closing the second panel based on detecting a second user selection to stop viewing one or more participants through the user interface.

8. The method of claim 6 or claim 7, further comprising:
opening the first panel based on detecting the first user selection to view one or more participants through the user interface.

9. The method of any preceding claim, wherein the second panel comprises a mute all control that inhibits an audio feed of the one or more participants of the second panel through the user interface.

10. The method of any preceding claim, wherein the selection of one of the one or more video streams of the second set comprises detecting a first selection of an expanded command menu and a second selection of a pin control on the expanded command menu.

11. A computer program product comprising a memory device having computer executable instructions stored thereon, which when executed by one or more processors cause the one or more processors to perform operations comprising:
displaying two or more video streams that include at least one video stream internal to an operating room on a panel of a display and at least one video stream external to the operating room on the panel of the display;
detecting a selection of one of the two or more video streams as a selected video stream;
moving a current video stream displayed in a main portion of the display to the panel based on detecting the selection and retaining a surgeon video stream position pinned at a same location on the panel before and after moving the current video stream from the main portion of the display to the panel; and
moving the selected video stream from the panel to the main portion of the display based on detecting the selection.

12. The computer program product of claim 11, wherein the at least one video stream internal to the operating room comprises at least one endoscopic video stream.

13. The computer program product of claim 12, wherein the operations further comprise:
maintaining the at least one endoscopic video stream at a position on the panel that has a higher viewing priority than the at least video stream external to the operating room on the panel.

14. The computer program product of claim 13, wherein the operations further comprise:
adjusting a relative position of one or more of the at least one video stream external to the operating room on the panel based on one or more placement rules.

15. The computer program product of any of claims 11 to 14, wherein the selection is separately controlled by each user interacting with the display.

16. The computer program product of claim 15, wherein the operations further comprise:
detecting a point-of-interest selection through at least one user interface of one of a plurality of users; and
generating an indication of the point-of-interest associated with at least one video stream of the display to note occurrence of an event captured in at least one of the video streams.

17. A system comprising:
an operating room display;
a memory system; and
a processing system coupled to the memory system and the operating room display, the processing system configured to execute a plurality of instructions to:
display a first set of one or more video streams that are internal to an operating room on a first panel of the operating room display;
display a second set of one or more video streams that are external to the operating room on a second panel of the operating room display;
detect a selection of one of the one or more video streams of the first set or the second set as a selected video stream; and
moving the selected video stream to the main portion of the operating room display based on detecting the selection.

18. The system of claim 17, wherein the first panel is arranged horizontally on the operating room display and the second panel is arranged vertically on the operating room display.

19. The system of claim 17 or claim 18, wherein the first panel and the second panel are opened based on detecting a user selection to view one or more participants through a user interface.

20. The system of any of claims 17 to 19, wherein the selection of one of the one or more video streams of the first set comprises a single user action, and the selection of one of the one or more video streams of the second set comprises at least two user actions.
